(19) United States
(12) Patent Application Publication
(10) Pub. No.: US 2004/0106854 A1
(43) Pub. Date: Jun. 3, 2004

(54) Telemeter for medical care

(75) Inventor: Yoshiya Muraki, Bunkyo-ku (JP)

Correspondence Address: OLIFF & BERRIDGE, PLC, P.O. BOX 19928, ALEXANDRIA, VA, 22320, US

(73) Assignee: Fukuda Denshi Co., Ltd., Tokyo (JP)

(21) Appl. No.: 10/305,964

(22) Filed: Nov. 29, 2002

(51) Int. Cl. A61B 5/00

(52) U.S. Cl. 600/300

(57) ABSTRACT

The present invention provides a telemeter for medical care to determine position information of a patient in a narrow range in the following manner. Position information transmitters using electromagnetic induction radio are arranged at necessary positions in a medical facility, and a receiver for receiving position information is built in the telemeter for medical care carried by each of patients. The position information is superimposed on a signal transmitted by the telemeter transmitter for medical care and a receiving device receives the position information together with the living body information of the patient. Further, the past position information of the patient is stored and compared with the present position information of the patient.

### BACKGROUND OF THE INVENTION

1. Field of the Invention

The present invention relates to a system of a telemeter for medical care.

2. Description of the Related Art

The telemeter for medical care is an apparatus that measures a living body phenomenon of a patient such as an electrocardiogram or the number of heart beats, amplifies the data of the living body phenomenon, transmits the data by means of a transmitter carried by the patient, receives the data in a nurse station and the like, and monitors the data on a display.

In Japanese Unexamined Patent Publication No. 10-248816 is disclosed a patient position detection system that includes infrared transmitters each of which is installed in each of a plurality of regions partitioned in a ward and transmits position information by the use of the infrared rays, a transmitter for medical care that is carried by the patient and receives the position information from the infrared transmitters and transmits the position information received from the infrared transmitters and identification information for identifying itself, and a monitor that receives radio waves transmitted by the transmitter for medical care by means of an antenna installed in the ward and demodulates the position information and the identification information from the received signals to display or record the position information and the identification information. Moreover, it is disclosed that the transmitter for medical care has a living body information measurement section for measuring an electrocardiogram and transmits the measurement results of the living body information measurement section together with the position information and the identification information.

Since this related art utilizes the infrared rays, it presents a problem that in a state where the infrared rays are blocked off, for example, under clothes or behind a pillar, a human body, furniture and the like, the position information may not be received. Moreover, since the related art neither stores the past position information of the patient nor compares the past position information of the patient with the present position information of the patient, it neither narrows down the position of the patient nor determines the direction of movement of the patient. For this reason, a large number of position information transmitters need to be installed.

Since the telemeter for medical care in the conventional art utilizes the infrared rays as means for transmitting the position information, in a case where the infrared rays can not be transmitted surely, for example, behind a human body, a thing, furniture, a pillar and the like, or under clothes, the position information can not be transmitted surely. Further, in a case where the vicinity of the position information transmitter is extremely light, for example, by a window in a daytime or under an operation light, the position information may not be transmitted. Still further, as to the position of the patient, the past position of the patient is neither stored nor compared with the present position information of the patient and thus the range of determining the position of the patient becomes wide and the direction of movement of the patient can not be determined. Still further, there is presented a problem that the determining the position of the patient narrowly results in increasing the number of position information transmitters. Still further, since the position information transmitter utilizing the infrared rays emits the infrared rays so as to cover a given range of space, for example, every nook and corner of a room, the infrared rays overlap those emitted by the neighboring position information transmitters and thus the position information received by the telemeter transmitter for medical care may interfere with the position information transmitted by the neighboring position information transmitters, which may result in receiving wrong information.

Moreover, in a case where electromagnetic induction radio transmitters installed in necessary positions to transmit the position information perform amplitude modulation including the position information for carrier waves, they may cause a trouble to a medical instrument carried by the patient such as a cardiac pacemaker, a hearing aid and the like.

### SUMMARY OF THE INVENTION

The object of the present invention is to provide a telemeter for medical care that solves the above-mentioned problems and has a function of determining the position of a patient with more accuracy.

The present invention is to solve the above-described problems by utilizing a character of directivity possessed by electromagnetic induction radio and storing the past position information of a patient and comparing the past position information with the present position information of the patient. The technical means of the present invention is a telemeter for medical care characterized in that it includes position information transmitters which use electromagnetic induction radio and are arranged at necessary positions in a medical facility; a receiver which is built in a telemeter transmitter for medical care and receives position information transmitted by the position information transmitters; a superimposing device which superimposes the position information on a signal transmitted by the telemeter transmitter for medical care; a receiver which receives the position information together with living body information; and a display device which displays the received living body information together with the received position information. Each of the position information transmitters transmits a signal peculiar to a position where the transmitter is arranged, for example, digital position information by means of electromagnetic induction radio.

This position information is received by a telemeter transmitter for medical care passing by the position designated by the position information. The received position information together with the received living body information are superimposed on the radio wave of the telemeter transmitter for medical care and are transmitted. This position information is received and stored by a center for receiving and monitoring the telemeter for medical care and is displayed together with the received living body information. The center for receiving and monitoring the telemeter for medical care stores and compares the past position information of a patient previously stored with the present position information of the patient and thus can narrow down the position range of the patient to a narrower range and determine also the direction of movement. Thus, this can minimize the number of position information transmitters to be installed.

When the above-mentioned present invention is made a telemeter for medical care characterized in that it further includes a storage device which stores the position information together with time information; a processing device which narrows down and pinpoints a present position and a direction of movement from the past position information previously stored and the newest position information; and a display device which displays the narrowed and pinpointed position together with the living body information, it is preferable that the telemeter for medical care can always get the present state of the patient.

Further, when the present invention is made a telemeter for medical care characterized in that the position information transmitter has a frequency modulator for modulating a carrier wave of the position information superimposed on electromagnetic induction radio transmitted by itself, it is preferable that the telemeter for medical care is in no danger of causing a trouble to a cardiac pacemaker, a hearing aid and the like.

The frequency modulating of a carrier wave of electromagnetic induction radio transmitted by the position information transmitter by the position information can make the energy of a transmitted electromagnetic wave a continuous uniform value. Therefore, this can reduce a possibility of causing a trouble to a medical instrument carried by the patient such as a cardiac pacemaker and a hearing aid and the like.

In a system in accordance with the present invention, the center for receiving and monitoring the telemeter for medical care can pinpoint a place where the patient carrying the telemeter transmitter for medical care is, so it is possible to give the patient an emergency medical treatment without delay.

### BRIEF DESCRIPTION OF THE DRAWINGS

FIG. 1 is a block diagram to show the overall configuration of a transmitting side of an embodiment.

FIG. 2 is a block diagram to show another embodiment of the transmitting side.

FIG. 3 is a block diagram to show an embodiment of a receiving side.

FIG. 4 is a plan view to illustrate a system of the present invention.

FIG. 5 is a flow chart for narrowing down position information to a narrow range.

### DESCRIPTION OF THE PREFERRED EMBODIMENT

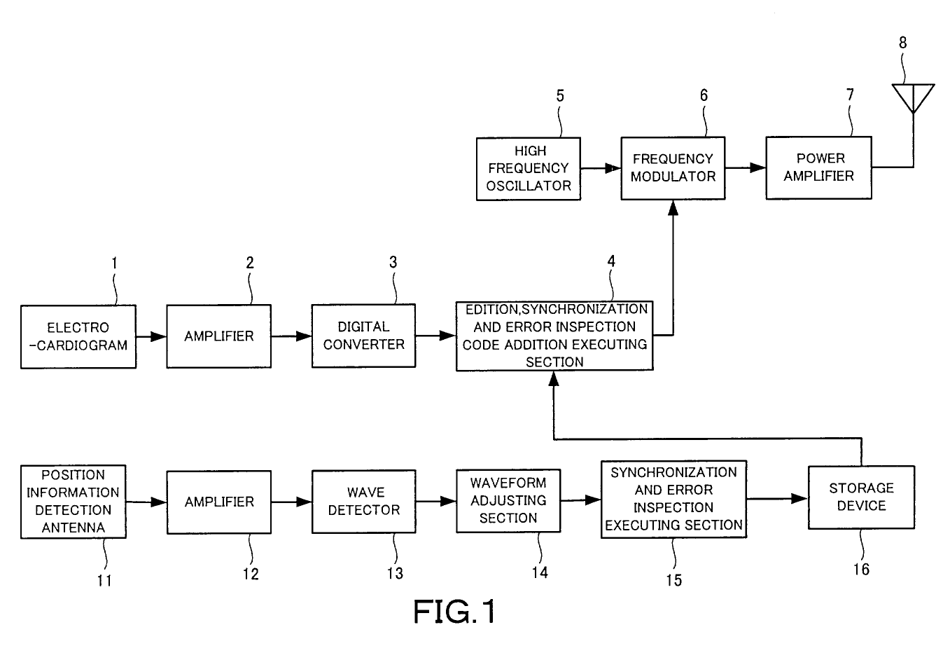

The embodiment of the present invention will be described with reference to the accompanying drawings. FIG. 1 is a block diagram to show the overall configuration of the transmitting side of a telemeter for medical care in accordance with an embodiment of the present invention. In the conventional art, an electrocardiogram 1 obtained by measuring a patient is amplified by an amplifier 2 and converted into digital data by a digital converter (A/D converter) 3. And then, edition, synchronization, and error inspection code addition is performed on this data by an edition, synchronization, and error inspection code addition executing section 4. On the other hand, a high frequency oscillator 5 generates a high frequency. The generated high frequency is modulated using a modulator 6 by superimposing the digital-converted data described above on the generated high frequency. The modulated data is transmitted from an antenna 8 by radio via a power amplifier 7.

According to the present invention, transmitters for transmitting position information are installed in necessary positions and transmit the position information. A device for receiving the position information is added to a transmitter carried by the patient. In the device for receiving the position information, the position information is received by a position information detection antenna 11, amplified by an amplifier 12, detected by a wave detector 13, adjusted by a waveform adjusting section 14. And then, synchronization and error inspection is performed on the position information by a synchronization and error inspection executing section 15, and the obtained data is stored in a storage device 16. The stored data is taken into a conventional telemeter transmitter for transmission to be subjected to edition, synchronization and error inspection code addition by the edition, synchronization and error inspection code addition executing section 4. Further, the data is superimposed on the high frequency together with the electrocardiogram information, modulated, and transmitted from the antenna 8.

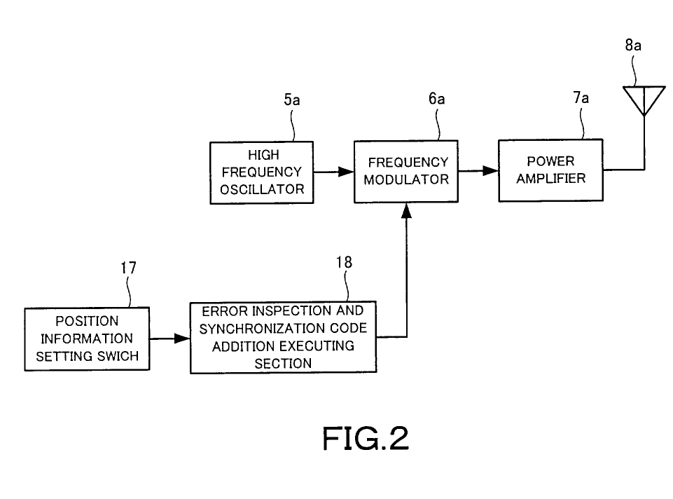

FIG. 2 shows an embodiment of a position information transmitting side. A position information setting switch 17 specifies position information, and error inspection and synchronization code addition is performed on the specified position information by an error inspection and synchronization code addition executing section 18. The position information is superimposed on a high frequency provided by a high frequency oscillator 5a, modulated by a frequency modulator 6a, power-amplified by a power amplifier 7a, and transmitted as electromagnetic induction radio from the antenna 8a.

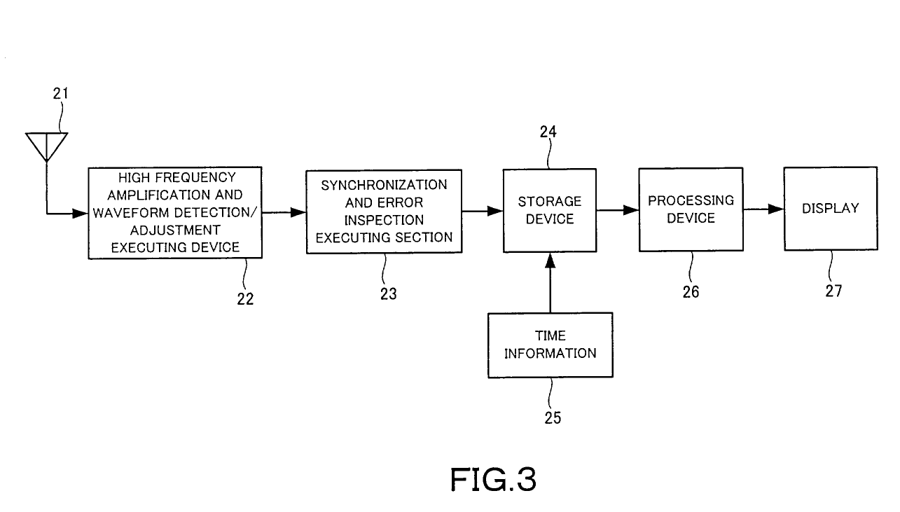

FIG. 3 is a block diagram of the receiving side of the telemeter for medical care of the embodiment. A receiving antenna 21 receives the radio waves of the telemeter transmitter for medical care. The received radio waveforms are processed by a high frequency amplification and waveform detection/adjustment executing device 22, subjected to synchronization and error inspection by a synchronization and error inspection executing section 23 and stored in a storage device 24. At this time, a clock device adds time information 25 of receipt. The information stored in the storage device 24 is processed by a processing device 26 and displayed as position information on a display 27. It is also possible to provide the processing device 26 with a function of narrowing down and determining the present position and the direction of movement based on the stored past position information and the latest position information to display the narrowed and pinpointed position on the display 27. Moreover, it is also possible to display on the display 27 living body information together with the position information or the living body information alone switched from the with the position information.

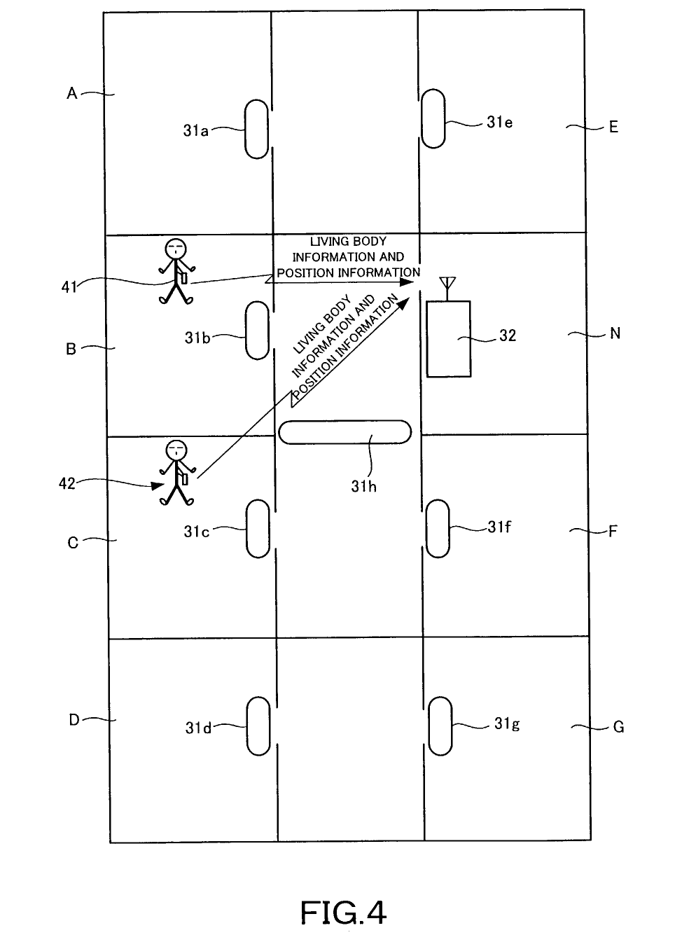

FIG. 4 is a plan view to illustrate a system in accordance with the present invention. At main positions designated by reference symbols A to G such as the doorways and the corridor of a ward are arranged position information transmitters 31a to 31h that transmit the position information at the respective positions. In a nurse station N are arranged a receiving device of a telemeter for medical care and a monitor 32. Each of patients 41, 42, . . . carries the telemeter transmitter for medical care. When an abnormality is found in the living body information of the patient that is received in the nurse station N, by changing a screen of the display device, the position display shown in FIG. 4 is displayed on the display, that is, the positions of the patients carrying the telemeter transmitters for medical care are displayed on the display. Therefore, it is possible to identify the position of the patient and to give the patient a necessary treatment immediately.

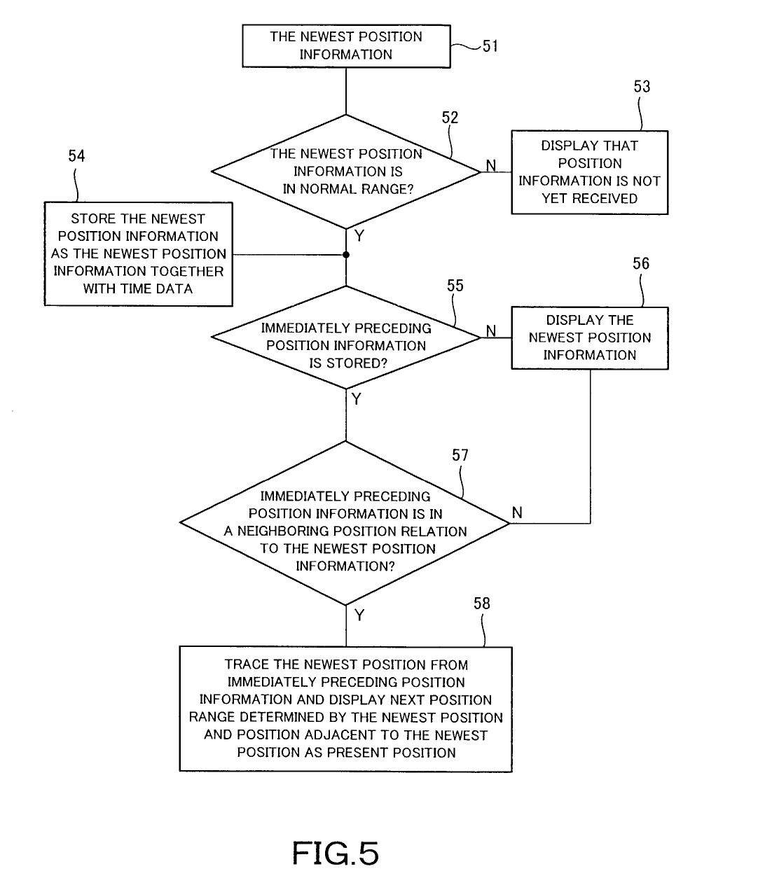

In FIG. 5 will be shown a flow chart to describe an operation of narrowing down the position information of the telemeter for medical care in accordance with the present invention. When the newest position information 51 is detected 51, it is judged whether or not the newest position information is position information in a normal range 52. If it is judged from the result of judgment in the step 52 that the newest position information is not position information in the normal range, it is displayed that the position information is not yet received 53. If it is judged in the step 52 that the newest position information is position information in the normal range, the newest position information is stored as the newest position information together with time data 54 and then it is judged whether or not the immediately preceding position information is stored 55. If it is judged to be NO as the result of judgment in the step 55, this position information is displayed as the newest position information 56. If it is judged to be YES as the result of judgment in the step 55, it is judged whether or not the immediately preceding position information is in a neighboring position relation to the newest position information 57. If it is judged to be NO as the result of judgment in the step 57, the newest position information is displayed 56. If it is judged to be YES as the result of judgment in the step 57, the newest position is traced from the immediately preceding position information, and the next position range determined by the newest position and a position adjacent to the newest position is displayed as the present position 58. By the procedures described above, according to the telemeter for medical care in accordance with the present invention, it is possible to always get the newest appropriate position information and further to narrow down the position to a narrow range and thus to give a patient an appropriate medical treatment immediately even when an emergency arises.

When an abnormality is found in a living body by the telemeter for medical care in the conventional art, sometimes, it is not possible to appropriately find the present position of the patient and to give the patient an emergency treatment. According to the present invention, however, it is possible to appropriately get the information such as the electrocardiogram and the like and the position information of the patient and thus to give the patient a necessary medical treatment immediately.

What is claimed is:

1. A telemeter for medical care comprising:
a position information transmitter using electromagnetic induction radio arranged at a necessary position in a medical facility;
a receiver that is built in a telemeter transmitter for medical care and receives position information transmitted by the position information transmitter;
a superimposing device that superimposes the position information on a signal transmitted by the telemeter transmitter for medical care;
a receiver that receives the position information together with living body information; and
a display device that displays the received living body information together with the received position information.

2. A telemeter for medical care as claimed in claim 1, further comprising:
a storage device that stores the position information together with time information;
a processing device that narrows down and pinpoints a present position and a direction of movement from the past position information previously stored and newest position information; and
a display device that displays the narrowed and pinpointed position together with the living body information.

3. A telemeter for medical care as claimed in claim 1, wherein the position information transmitter has a frequency modulator that modulates a carrier wave of the position information superimposed on electromagnetic induction radio transmitted by itself.

4. A telemeter for medical care as claimed in claim 2, wherein the position information transmitter has a frequency modulator that modulates a carrier wave of the position information superimposed on electromagnetic induction radio transmitted by itself.
